(19) United States
(12) Patent Application Publication
(10) Pub. No.: US 2017/0052202 A1
(43) Pub. Date: Feb. 23, 2017

(54) Methods for Diagnosing and Treating Iron Dysregulation

(71) Applicant: INSERM (Institut National de la Sante et de la Recherche Medicale), Paris (FR)

(72) Inventor: Marie-Paule Roth, Toulouse Cedex (FR)

(21) Appl. No.: 15/344,197

(22) Filed: Nov. 4, 2016

Related U.S. Application Data
(63) Continuation of application No. 14/045,531, filed on Oct. 3, 2013, now abandoned.
Application No. 14/045,531 is a continuation of application No. 12/868,449, filed on Aug. 25, 2010, now abandoned.
(60) Provisional application No. 61/238,737, filed on Sep. 1, 2009.

(51) Int. Cl. G01N 33/74; C12Q 1/68; C07K 14/51

(52) U.S. Cl. CPC G01N 33/74; C07K 14/51; C12Q 1/6883; A61K 38/00

(57) ABSTRACT

The present invention relates to methods for diagnosing and treating iron overload and iron deficiency.

### RELATED APPLICATIONS

The present application is filed as a continuation application of U.S. patent application Ser. No. 14/045,531 filed Oct. 3, 2013, which is a continuation application of U.S. patent application Ser. No. 12/868,449 filed Aug. 25, 2010, which claims the benefit of priority of U.S. Provisional Patent Application No. 61/238,737, which was filed Sep. 1, 2009. The entire text of the aforementioned application is incorporated herein by reference in its entirety.

### FEDERALLY SPONSORED RESEARCH OR DEVELOPMENT

[Not Applicable]

### FIELD OF THE INVENTION

The present invention relates to methods for diagnosing and treating iron overload and iron deficiency.

### BACKGROUND OF THE INVENTION

Iron is a key component of oxygen-transporting storage molecule, such as haemoglobin and myoglobin. Iron deficiency results in anemia, while iron overload leads to tissue damage and fibrosis.

Hepcidin is a peptide hormone produced by hepatocytes and is a negative regulator of iron entry into plasma. Hepcidin acts by binding to cellular iron exporter ferroportin, present on cells of the intestinal duodenum and macrophages. Hepcidin induces the endocytosis and proteolysis of ferroportin, preventing release of iron from intestinal cells and macrophages into the plasma.

Hemochromatosis is a disease caused by inappropriate iron absorption and is one of the most common autosomal recessive diseases affecting populations of north European origin. In genetic hemochromatosis, sustained deficiency of hepcidin causes excessive iron absorption from the diet and leads to the deposition of iron in the liver and other tissues. Iron plays a key role in the formation of toxic oxygen radicals, leading to consequent organ damages and functional failures.

The most widely used treatment for hemochromatosis is phlebotomy. This method of treatment present many shortcomings: the amount of iron removed per phlebotomy is limited and the number of phlebotomies an individual is able to tolerate is also limited. Furthermore, the procedure of phlebotomy is really restrictive for the treated patient. There is thus a need for a more easily acceptable treatment for iron dyresgulation.

### BRIEF SUMMARY OF THE INVENTION

The inventors have shown for the first time a previously unexpected but essential role of BMP6 in the maintenance of iron homeostasis. The expression of hepcidin, which is a negative regulator of iron entry into plasma, is regulated by BMP6. The inventors also have shown that BMP6 levels are high in untreated iron loaded hemochromatosis patients and that removing excess iron stores lead to a decrease in hepcidin expression, which explain the reaccumulation of iron in these patients.

The present invention provides a method for diagnosing an iron dysregulation in a subject, said method comprising the step of measuring the level of BMP6 in a body fluid such as whole blood, blood plasma, serum or urine obtained from said subject.

The present invention provides a method for preventing iron accumulation in a subject, comprising the step of administering to said subject an effective amount of BMP6, a fragment or a derivative thereof which induces hepcidin expression or a vector comprising a nucleic acid coding for BMP6, a fragment or a derivative thereof which induces hepcidin expression.

The present invention also provides a method for preventing iron reaccumulation in a subject who has been iron depleted, said method comprising the step of administering to said subject an effective amount of BMP6, a fragment or a derivative thereof which induces hepcidin expression or a vector comprising a nucleic acid coding for BMP6, a fragment or a derivative thereof which induces hepcidin expression.

The present invention provides a method for treating a subject suffering from iron deficiency, said method comprising the step of administering to said subject an effective amount of an inhibitor of BMP6 induction of hepcidin expression to said subject.

The present invention further provides a method for diagnosing an autosomal recessive hereditary pathology, or a risk of an autosomal recessive hereditary pathology, in a subject, said method comprising detecting homozygosity or compound heterozygosity for defective mutation(s) in the BMP6 gene in a sample obtained from said subject, wherein the presence of two homozygous defective BMP6 mutations is indicative of an autosomal recessive pathology or a risk of an autosomal recessive hereditary pathology.

### BRIEF DESCRIPTION OF SEVERAL VIEWS OF THE DRAWINGS

FIG. 1: Effect of Bmp6-deficiency on serum transferrin saturation, hepatic and splenic iron concentrations, hepcidin gene expression, and hepcidin response to LPS treatment.

(a) Transferrin saturation (%) and non-heme tissue iron content (μg/g wet wt) were compared in wild-type mice (WT) and in Bmp6-deficient mice (Bmp6^{-/-}) at 7 weeks of age. Means of six samples±SE are represented on this figure. Student's t-tests were performed on log-transformed values of tissue iron concentrations.

(b) Expression ratio (and standard error) of Hamp1 transcripts in Bmp6^{-/-} mice relative to wild-type controls (6 mice per group) and normalized to the reference gene mRNA (beta-glucuronidase) was calculated using the relative expression software REST. Statistical significance was determined using randomization tests.

(c) Expression ratios (and standard errors) of Hamp1 transcripts in WT mice treated by LPS, Bmp6^{-/-} mice treated with 0.9% NaCl, and Bmp6^{-/-} mice treated with LPS, relative to WT mice treated with 0.9% NaCl, and normalized to the reference gene mRNA (beta-glucuronidase) were calculated as in (b).

FIG. 2: Histological examination of iron loading. Tissue iron was detected by staining with Perls Prussian blue (blue stain). (a) Wild-type liver. (b) Bmp6^{-/-} liver. (c) Wild-type spleen. (d) Bmp6^{-/-} spleen. Original magnification, ×200.

FIG. 3: Lack of significant phospho-Smad1/5/8 staining in the hepatocyte nuclei of Bmp6-deficient mice. Tissue sections were stained with anti-pSmad1/5/8 antibody and a green-fluorescent Alexa Fluor 488 secondary antibody. Nuclei were stained with propidium iodide (PI). Liver sections of (a) Bmp6^{-/-} mice fed a diet of normal iron content, (b) wild-type mice fed a diet of normal iron content, and (c) wild-type mice fed an iron-enriched diet.

FIG. 4: Increased Dmt1 and ferroportin expression in the proximal duodenum of Bmp6-deficient mice. DMT1 expression was detected by immunochemistry in (a) wild-type and (b) Bmp6^{-/-} mice. Bmp6^{-/-} mice have intense staining along the brush border. Ferroportin expression was detected by immunochemistry in (c) wild-type and (d) Bmp6^{-/-} mice. In mutant animals, staining is expressed intensely along the basolateral membrane of the enterocytes of the distal two-thirds of the villus. Original magnification, ×200 (a and b), ×400 (c and d).

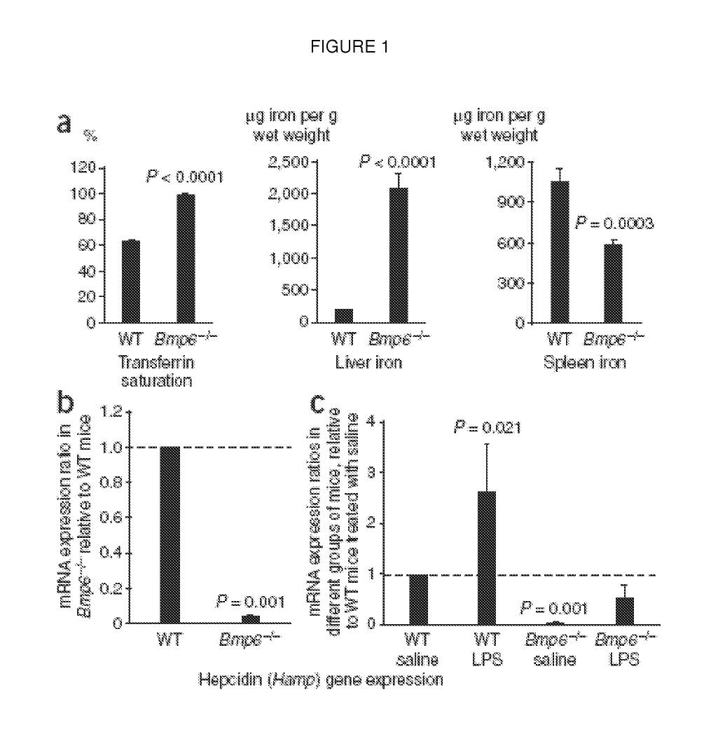

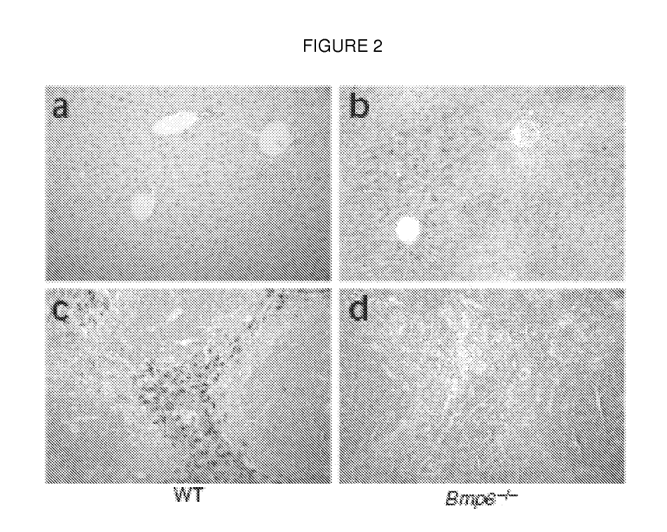

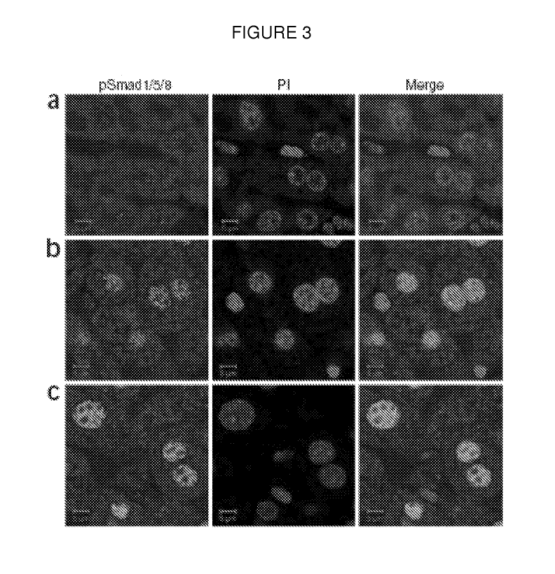

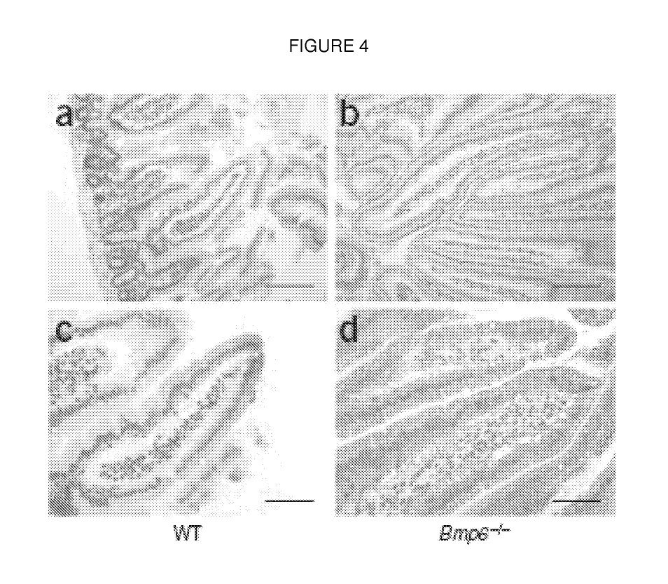

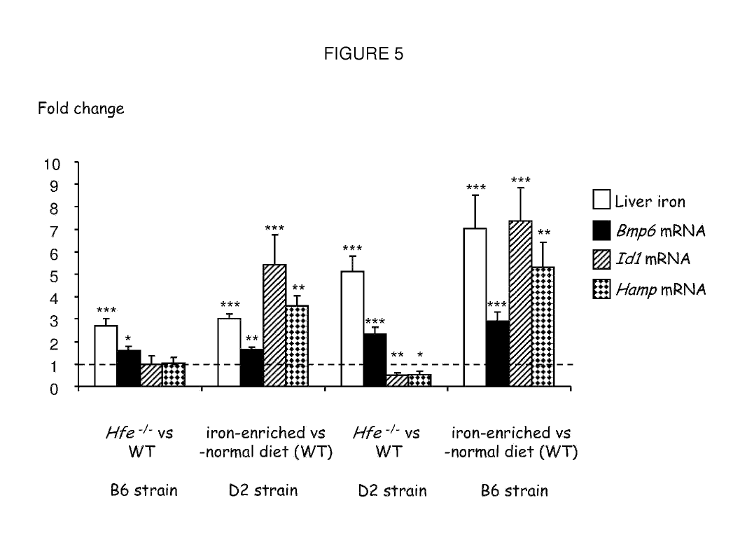

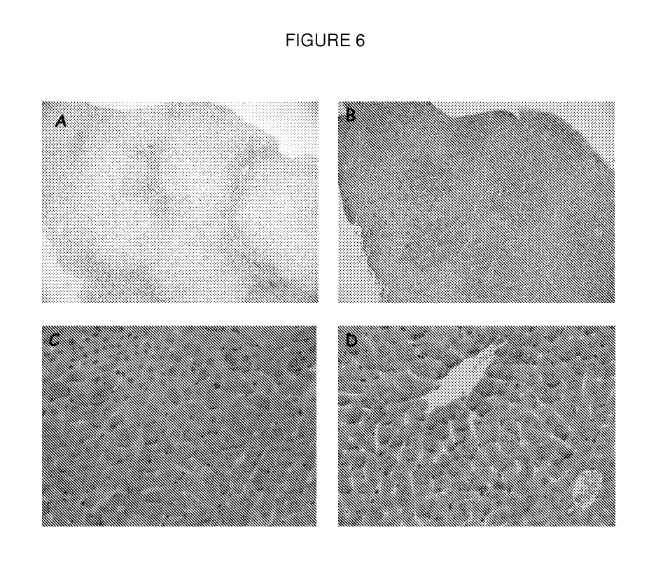

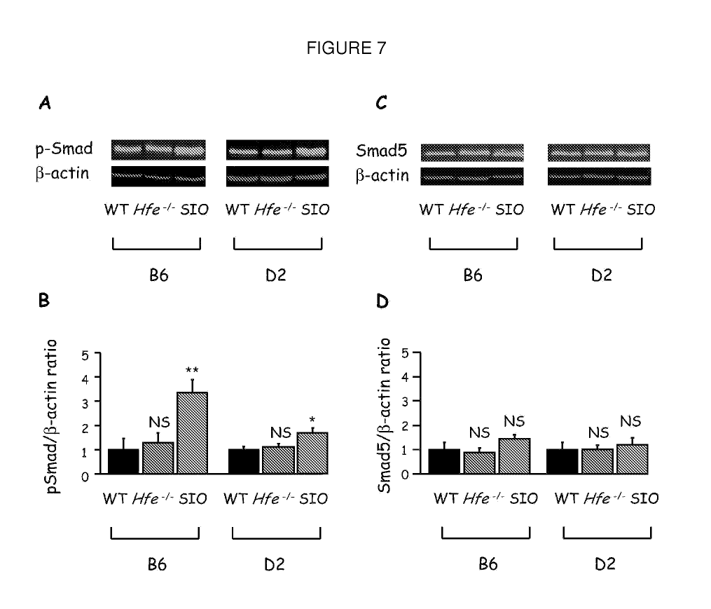

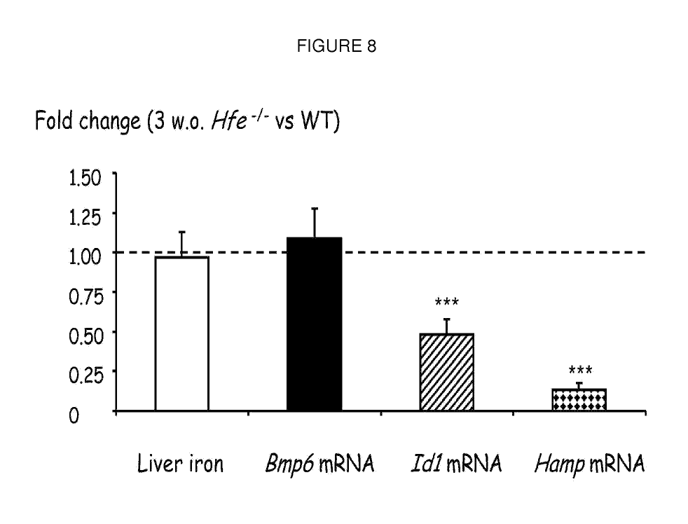

FIG. 5: Effect of Hfe-deficiency or secondary iron overload on hepatic iron concentrations and Bmp6, Id1, and Hamp gene expression in 7 week-old B6 and D2 mice. Fold-change in non-heme tissue iron content and expression ratio (and standard error) of Bmp6, Id1, and Hamp transcripts normalized to the reference gene mRNA (Hprt) in Hfe-deficient mice relative to wild-type controls and in wild-type mice fed an iron-rich diet for three weeks relative to wild-type mice fed a standard rodent diet (5-10 mice per group). Statistical significance was determined using randomization tests. *P<0.05; **P<0.01; ***P<0.001. Data are provided for two genetic backgrounds, C57BL/6 (B6) and DBA/2 (D2). At 7 weeks of age, wild-type mice of the two backgrounds have similar levels of Bmp6, Id1 and Hamp transcripts (see Supplemental FIG. 1). However, Hfe-deficient mice of the D2 background have significantly more Bmp6 mRNA than Hfe-deficient mice of the B6 background (p=0.001). Wild-type B6 mice fed the iron-rich diet for three weeks also have significantly more Bmp6 mRNA than wild-type D2 mice fed the same diet (p=0.001).

FIG. 6: Cellular localization of BMP6 in hepatic iron overload. BMP6 expression was detected by immunohistochemistry in (B and C) wild-type B6 mice with secondary iron overload, and (D) Hfe-deficient D2 mice. These mice have similar degrees of iron loading. As can be seen in serial liver sections, whereas iron deposits visualized by Perls staining (A) are predominantly periportal, BMP6 staining is mostly centrilobular (B). Mutant animals and mice with secondary iron overload have intense staining at the basolateral membrane domain of hepatocytes (C and D). Original magnification, ×100 (A and B) or ×400 (C and D).

FIG. 7: Smad1/5/8 phosphorylation is increased by secondary iron overload but unchanged by Hfe-deficiency.

(A) Liver lysates from wild-type controls fed a standard rodent diet (WT), Hfe-deficient mice (Hfe^{-/-}) and mice with secondary iron overload (SIO) were analyzed by western blot with antibodies to phosphorylated Smad1/5/8 and to β-actin as loading control. Membranes were scanned on the Odyssey Infrared Imaging System. One representative experiment is shown for each strain.

(B) Band sizing was performed using the Odyssey 3.0 software (LI-COR Biosciences) and quantification of phosphorylated Smads was calculated by normalizing the specific probe band to β-actin. Mean ratio (p-Smad/β-actin) of three Hfe-deficient mouse samples (or three mice with secondary iron overload)±SE are represented on this figure, relative to the mean ratio of three wild-type mice fed a standard rodent diet. Student's t-tests were used to compare mean ratios between Hfe-deficient mice and wild-type controls (p=0.55 for B6 mice; p=0.58 for D2 mice) or between mice with secondary iron overload and wild-type mice (**p=0.01 for B6 mice; *p=0.02 for D2 mice).

(C) Liver lysates from the same mice were analyzed by western blot with antibodies to Smad5 and to β-actin as loading control.

(D) Quantification using the Odyssey 3.0 software was performed as in (B). Student's t-tests were used to compare mean Smad5/β-actin ratios. The levels of Smad5 were not significantly different between Hfe-deficient mice and wild-type controls (p=0.59 for B6 mice; p=0.59 for D2 mice), or between mice with secondary iron overload and wild-type controls (p=0.15 for B6 mice; p=0.31 for D2 mice).

FIG. 8: Effect of Hfe-deficiency on hepatic iron concentrations and Bmp6, Id1, and Hamp gene expression in 3 week-old mice.

Fold-change in non-heme tissue iron content and expression ratio (and standard error) of Bmp6, Id1, and Hamp transcripts normalized to the reference gene mRNA (Hprt) in 3 w.o. Hfe-deficient mice relative to wild-type controls (8 mice per group). Statistical significance was determined using randomization tests. ***p<0.001. At 3 weeks of age, wild-type mice have levels of Bmp6 and Id1 mRNAs similar to 7 week-old mice. Although they have slightly less Hamp gene expression than 7 week-old mice, the difference is not statistically significant.

### DETAILED DESCRIPTION OF THE INVENTION

### Definitions

As used herein, “iron dysregulation” is intended to include both iron overload and iron deficiency.

As used herein, “iron overload” refers to diseases, syndromes, or conditions resulting in an iron overload in a patient, including but not limited to primary iron overload such as adult and juvenile forms of hemochromatosis resulting from mutations in the HFE, TFR2, FPN, HJV, or BMP6 gene, as well as secondary iron overload resulting from thalassemias, sideroblastic anemias, dietary iron overload, long term haemodialysis, chronic liver disease or dysmetabolic iron overload syndrome.

As used herein, “iron deficiency” refers to diseases, syndromes, or conditions resulting in an iron deficiency in a patient, including but not limited to iron deficiency anemia and anemia of chronic disease, a prevalent condition that afflicts patients with a wide variety of diseases, including infections, malignancies and rheumatologic disorders.

As used herein, “detection” includes qualitative and/or quantitative detection (measuring levels) with or without reference to a control. Typically, BMP6 level can be measured by chromatography, especially cation-exchange chromatography, electrophoresis, chemiluminescence, immunodot by anti-BMP6 antibody, and enzyme linked immunoassay for BMP6. Those assays accurately enable the measurement of BMP6 levels in body fluids such as whole blood, blood plasma, serum or urine.

Method of Diagnostic

The invention relates to a method for diagnosing an iron dysregulation in a subject comprising the step of measuring the level of BMP6 in a body fluid such as whole blood, blood plasma, serum or urine obtained from said subject.

A high BMP6 level is associated with an iron deficiency. A low BMP6 level is associated with an iron overload. Such levels are compared to a physiological normal level of BMP6, which can be easily determined by the skilled man.

Typically, the level of expression of BMP6 can be measured by chromatography, especially cation-exchange chromatography, electrophoresis, chemiluminescence, immunodot by anti-BMP6 antibody, and enzyme linked immunoassay for BMP6. It falls within the ability of the skilled man to carry out such methods.

The method of the invention may be used in combination with traditional methods used to diagnose iron dysregulation in a subject. Typically, a physician may also consider other clinical or pathological parameters used in existing methods to diagnose iron dysregulation. Thus, results obtained using the method of the present invention may be compared to and/or combined with results from other tests, assays or procedures performed for the diagnosis of iron dysregulation. Such comparison and/or combination may help provide a more refine diagnosis.

Prevention from Iron Overload

The present invention provides a method for preventing iron accumulation in a subject, comprising the step of administering to said subject:
an effective amount of BMP6, a fragment or a derivative thereof, said fragment or derivative inducing hepcidin expression; or
an effective amount of a vector comprising a nucleic acid coding for BMP6, a fragment or a derivative thereof, said fragment or derivative inducing hepcidin expression.

In another embodiment of the invention, the subject is predisposed to iron overload. Examples of predisposition to iron overload are genetic predisposition related to a mutation of the HFE, TFR2, HJV or BMP6 gene.

The present invention also provides a method for preventing iron reaccumulation in a subject who has been iron depleted, said method comprising the step of administering to said subject:
an effective amount of BMP6, a fragment or a derivative thereof, said fragment or derivative inducing hepcidin expression; or
an effective amount of a vector comprising a nucleic acid coding for BMP6, a fragment or a derivative thereof, said fragment or derivative inducing hepcidin expression.

Typically, said subject is iron depleted by phlebotomy. As used herein, “phlebotomy” refers to a surgical incision into a vein in order to remove blood for treatment purposes.

Typically, BMP6, a fragment or a derivative thereof, or a vector comprising a nucleic acid coding for BMP6, a fragment or a derivative thereof may be used for treating a subject suffering from iron overload, preventing iron overload in a subject predisposed to genetic hemochromatosis or preventing iron reaccumulation after phlebotomies.

Typically, BMP6 fragments comprise regions of BMP6 amino acid sequence having a length comprised between 30 to 140, preferably between 50 and 130, and more preferably between 70 and 120 amino acids and which induce hepcidin expression.

Typically, BMP6 derivatives comprise an amino acid sequence comprising at least 95%, preferably at least 96%, 97% or 98%, and more preferably at least 99% amino acid sequence identity over BMP6 amino acid sequence, and induces hepcidin expression. Such BMP6 derivatives may contain deletions, additions, or substitutions of amino acid residues within the BMP6 amino acid sequence.

Typically, the vector of the invention can be a viral vector or a plasmid used to introduce a nucleic acid coding for BMP6, a fragment or a derivative thereof which induce hepcidin expression. Typically, the vector comprising the nucleic acid coding for BMP6 is a viral vector specifically targeted to a desired cell or tissue, e.g. hepatocytes. Specific targeting may be conferred by incorporation of an affinity binding molecule into the vector envelope that has a cognate binding molecule present on the surface of the targeted cells or tissue. Specific targeting may also be conferred by incorporating a liver specific promoter into the vector coding for BMP6, a fragment or a derivative thereof, so that the expression of BMP6 is induced in hepatocytes.

By an “effective amount of BMP6, a fragment or a derivative thereof” is meant a sufficient amount to treat iron overload, at a reasonable benefit/risk ratio applicable to any medical treatment. It will be understood, however, that the total daily usage of BMP6, a fragment or a derivative thereof will be decided by attending physician within the scope of sound medical judgment. The specific therapeutically effective dose level for any particular subject in need thereof will depend upon a variety of factors including the stage of iron overload being treated and the activity of the specific BMP6, the fragment or the derivative thereof or the vector comprising a nucleic acid coding for BMP6 employed, the age, body weight, general health, sex and diet of the subject, the time of administration, route of administration, the duration of the treatment, drugs used in combination or coincidental with the treatment.

By “percent (%) amino acid sequence identity” is meant the percentage of amino acid residues in a candidate sequence that are identical with the amino acid residues in a BMP6 sequence, after aligning the sequences and introducing gaps, if necessary, to achieve the maximum percent sequence identity, and not considering any conservative substitutions as part of the sequence identity.

Treatment of Iron Deficiency

The present invention further relates to a method for treating a subject suffering from iron deficiency, comprising the step of administering to said subject an effective amount of an inhibitor of BMP6 induction of hepcidin expression.

By an “effective amount of an inhibitor of BMP6 induction of hepcidin expression” is meant a sufficient amount to treat iron deficiency, at a reasonable benefit/risk ratio applicable to any medical treatment. It will be understood, however, that the total daily usage of inhibitor of BMP6 induction of hepcidin expression will be decided by attending physician within the scope of sound medical judgment. The specific effective dose level for any particular subject in need thereof will depend upon a variety of factors including the stage of iron deficiency being treated and the activity of the specific inhibitor of BMP6 induction of hepcidin expression employed, the age, body weight, general health, sex and diet of the subject, the time of administration, route of administration, the duration of the treatment, drugs used in combination or coincidental with the treatment.

The expression “inhibitor of BMP6 induction of hepcidin expression” should be understood broadly, the expression refers to agents downregulating the expression of BMP6 or compounds that bind to BMP6 and inhibit its activity.

Typically, the inhibition of BMP6 induction of hepcidin expression can be measured by chromatography, especially cation-exchange chromatography, electrophoresis, chemiluminescence, immunodot by anti-hepcidin antibody, and enzyme linked immunoassay for hepcidin (see for example Ganz T et al, Immunoassay for human serum hepcidin, Blood 2008, 112:4292-7).

In an embodiment of the present invention, the inhibitor of BMP6 activity is an agent downregulating BMP6 induction of hepcidin expression. Typically, agents downregulating BMP6 induction of hepcidin expression comprise a nucleic acid which interferes with the expression of BMP6.

Examples of such agents are antisense molecules or vectors comprising said antisense molecules. Antisense molecules are complementary strands of small segments of mRNA. Methods for designing effective antisense molecules being well known (see for example U.S. Pat. No. 6,165,990), it falls within the ability of the skilled artisan to design antisense molecules able to downregulate the expression of BMP6 in hepatocytes. Further examples are RNA interference (RNAi) molecules such as, for example, short interfering RNAs (siRNAs) and short harpin RNAs (shRNAs). RNAi refers to the introduction of homologous double stranded RNA to specifically target a gene's product, in the present case BMP6, resulting in a null or hypomorphic phenotype. Methods for designing effective RNAi molecules being well known (see for review Hannon and Rossi Nature. 2004 Sep. 16; 431 (70006): 371-8) it falls within the ability of the skilled artisan to design RNAi molecules able to down-regulate BMP6 induction of hepcidin expression in hepatocytes.

In a further embodiment of the invention, the inhibitor of BMP6 induction of hepcidin expression is an antibody against BMP6 or a fragment or derivative thereof which inhibits BMP6 induction of hepcidin expression. The person skilled in the art will be aware of standard methods for production of such specific antibody. For example, specific antibodies or biologically active fragments or derivatives thereof may be generated by immunizing an animal, for example KO BMP6 mice, with BMP6 or BMP6 fragments and by selecting the antibodies which bind to BMP6 and inhibit BMP6 induction of hepcidin expression.

The person skilled in the art will be aware of standard methods for production of both polyclonal and monoclonal antibodies and biologically active fragments and derivatives thereof. By biologically active fragment or derivative thereof, it is meant able to bind to the same epitope as the antibody, in the present case an epitope of BMP6, and able to inhibit BMP6 induction of hepcidin expression. Antibody fragments, particularly Fab fragments and other fragments which retain epitope-binding capacity and specificity are also well known, as are chimeric antibodies, and “humanized” antibodies, in which structural (not determining specificity for antigen) regions of the antibody are replaced with analogous or similar regions from another species. Thus antibodies generated in mice can be “humanized” to reduce negative effects which may occur upon administration to human subjects. The present invention therefore comprehends use of antibody specific for BMP6 which include F(ab′)₂, F(ab)₂, Fab, Fv and Fd antibody fragments, chimeric antibodies in which one or more regions have been replaced by homologous human or non-human portions. The person skilled in the art will also be aware that biologically active antibody derivatives such as for example ScFv fragments and divalent ScFv-type molecules can be prepared using recombinant methods.

The present invention also relates to a medicament comprising an inhibitor of BMP6 induction of hepcidin expression together with a pharmaceutically acceptable carrier. A person skilled in the art will be aware of suitable carriers. Suitable formulations for administration by any desired route may be prepared by standard methods, for example by reference to well-known text such as Remington; The Science and Practice of Pharmacy.

Inhibitors of BMP are well known. Typically, such inhibitors are for example the members of the cystine knot family of BMP antagonist, such as the protein Noggin (see Groppe et al, Stuctural basis of BMP signalling inhibition by the cystine knot protein Noggin, Nature 2002, 420:636-642).

Method for Diagnosing Autosomal Recessive Hereditary Pathology

The invention also relates to a method for diagnosing an autosomal recessive hereditary pathology, or a risk of an autosomal recessive hereditary pathology, in a subject, said method comprising the step of detecting a defective mutation in the BMP6 gene in a sample obtained from said subject, wherein the presence of homozygosity or compound heterozygosity for BMP6 mutations is indicative of an autosomal recessive pathology or a risk of an autosomal recessive hereditary pathology.

In a preferred embodiment, the defective mutation in the BMP6 gene is a mutation which results in a reduction of BMP6 expression or in impaired binding to type 1 and type 2 receptors. Said mutation may be detected by analyzing a BMP6 nucleic acid molecule. In the context of the invention, BMP6 nucleic acid molecules include mRNA, genomic DNA and cDNA derived from mRNA. DNA or RNA can be single stranded or double stranded. These may be used for detection by amplification and/or hybridization with a probe, for instance.

The nucleic acid sample may be obtained from any cell source or tissue biopsy. Non-limiting examples of cell sources available include without limitation blood cells, buccal cells, epithelial cells, fibroblasts, or any cells present in a tissue obtained by biopsy. Cells may also be obtained from body fluids, such as blood, plasma, serum, lymph, etc. DNA may be extracted using any methods known in the art, such as described in Sambrook et al., 1989. RNA may also be isolated, for instance from tissue biopsy, using standard methods well known to the one skilled in the art such as guanidium thiocyanate-phenol-chloroform extraction.

BMP6 mutations may be detected in a RNA or DNA sample, preferably after amplification. For instance, the isolated RNA may be subjected to coupled reverse transcription and amplification, such as reverse transcription and amplification by polymerase chain reaction (RT-PCR), using specific oligonucleotide primers that are specific for a mutated site or that enable amplification of a region containing the mutated site. According to a first alternative, conditions for primer annealing may be chosen to ensure specific reverse transcription (where appropriate) and amplification; so that the appearance of an amplification product be a diagnostic of the presence of a particular BMP6 mutation. Otherwise, RNA may be reverse-transcribed and amplified, or DNA may be amplified, after which a mutated site may be detected in the amplified sequence by hybridization with a suitable probe or by direct sequencing, or any other appropriate method known in the art. For instance, a cDNA obtained from RNA may be cloned and sequenced to identify a mutation in BMP6 sequence.

Actually numerous strategies for genotype analysis are available (Antonarakis et al., 1989; Cooper et al., 1991; Grompe, 1993). Briefly, the nucleic acid molecule may be tested for the presence or absence of a restriction site. When a base substitution mutation creates or abolishes the recognition site of a restriction enzyme, this allows a simple direct PCR test for the mutation. Further strategies include, but are not limited to, direct sequencing, restriction fragment length polymorphism (RFLP) analysis; hybridization with allele-specific oligonucleotides (ASO) that are short synthetic probes which hybridize only to a perfectly matched sequence under suitably stringent hybridization conditions; allele-specific PCR; PCR using mutagenic primers; ligase-PCR, HOT cleavage; denaturing gradient gel electrophoresis (DGGE), temperature denaturing gradient gel electrophoresis (TGGE), single-stranded conformational polymorphism (SSCP) and denaturing high performance liquid chromatography (Kuklin et al., 1997). Direct sequencing may be accomplished by any method, including without limitation chemical sequencing, using the Maxam-Gilbert method; by enzymatic sequencing, using the Sanger method; mass spectrometry sequencing; sequencing using a chip-based technology; and real-time quantitative PCR. Preferably, DNA from a subject is first subjected to amplification by polymerase chain reaction (PCR) using specific amplification primers. However several other methods are available, allowing DNA to be studied independently of PCR, such as the rolling circle amplification (RCA), the InvaderTMassay, or oligonucleotide ligation assay (OLA). OLA may be used for revealing base substitution mutations. According to this method, two oligonucleotides are constructed that hybridize to adjacent sequences in the target nucleic acid, with the join sited at the position of the mutation. DNA ligase will covalently join the two oligonucleotides only if they are perfectly hybridized.

In the following, the invention will be illustrated by means of the following examples as well as the figures.

### EXAMPLE

### Example 1

### Lack of BMP6 Induces Iron Overload

Summary

Expression of hepcidin, a key regulator of intestinal iron absorption, can be induced in vitro by several bone-morphogenetic proteins, including BMP2, BMP4, and BMP9. However, in contrast to BMP6, gene expression of other BMPs is not regulated by iron in vivo and their relevance to iron homeostasis is unclear. The inventors have shown that targeted disruption of Bmp6 in mice causes a rapid and massive accumulation of iron in the liver, in acinar cells of the exocrine pancreas, the heart, and renal convoluted tubules. In spite of their severe iron overload, the livers of Bmp6-deficient mice have low levels of phosphorylated Smads 1, 5 and 8, and these Smads are not significantly translocated to the nucleus. Hepcidin synthesis is markedly reduced. This demonstrates that Bmp6 is critical for iron homeostasis and that it is functionally nonredundant with other members of the Bmp subfamily. Of note, Bmp6-deficient mice retain their capacity to induce hepcidin in response to inflammation. The iron burden in Bmp6 mutant mice is significantly greater than in Hfe-deficient mice, indicating that mutations in the BMP6 gene cause iron overload in patients with severe juvenile hemochromatosis.

Methods

Mice

Bmp6 null mice (Bmp6^{m1Rob}) were derived as previously described and maintained on a background derived from the same stock (outbred CD1) as wild-type controls. The targeted allele was confirmed to encode a loss-of-function mutation. The inventors checked that insertion of the Neomycin resistance selection marker in exon 2 of Bmp6 had not caused deficiency of Txndc5, a gene adjacent to Bmp6, in the liver of these mice. All experiments were performed on males. Unless otherwise specified, mice received a diet of normal iron content (200 mg iron/kg; SAFE, Augy, France) and were analysed at 7 weeks and fasted for 14 h before they were killed. Experimental iron overload was obtained by feeding wild-type controls an iron-enriched diet (8.5 g/kg). Experimental protocols were approved by the Midi-Pyrénées Animal Ethics Committee.

LPS Injection

LPS (1 μg/g body weight; serotype 055:135; Sigma, Saint-Louis, Mich.) or an equivalent volume of saline was injected intraperitonally and organs (liver and spleen) were isolated 6 hours after injection.

Tissue Iron Staining and Quantitative Tissue Iron Measurement.

Intestine, liver, spleen, heart, pancreas and kidney samples were fixed in 10% buffered formalin and embedded in paraffin. Deparaffinized tissue sections were stained with the Perls Prussian blue stain for non-heme iron and counterstained with nuclear fast red. Quantitative measurement of non-heme iron in the liver and the spleen was performed as described previously. Results are reported as micrograms of iron per gram wet weight of tissue.

Immunohistochemistry

Four-micrometer sections of paraffin-embedded tissues were mounted on glass slides. Endogenous peroxidase activity was quenched by incubating specimens with Peroxisase Block (Dakocytomation EnVision+ System-HRP, Dako, Trappes, France). Sections were blocked in PBS containing 1% BSA and 10% foetal calf serum (Invitrogen, Paisley, UK) and incubated 1 h at RT with the primary anti-DMT1 antibody or 1 h30 at 37° C. with the primary anti-ferroportin antibody diluted in PBS-1% BSA. Immunohistochemical staining was performed using the DakoCytomation EnVision+ System-HRP according to the instructions of the manufacturer. Sections were counterstained with hematoxylin.

Immunofluorescence

Nonspecific fluorescence due to endogenous avidin and biotin was blocked by the Avidin Biotin Blocking Solution (Lab Vision, Fremont, Calif.). After permeabilization with 0.3% Triton X-100 in PBS-5% BSA, liver tissue sections were incubated with a rabbit polyclonal antibody to phosphorylated Smad1/5/8 (1/100; Cell Signaling Technology, Danvers, Mass.) overnight at 4° C. Staining was obtained using the Alexa Fluor 488 goat anti-rabbit secondary antibody (1/200; Invitrogen) and slides were mounted in VECTASHIELD Mounting Medium containing propidium iodide (Clinisciences, Montrouge, France) to counterstain DNA. Cells were visualized using a Zeiss confocal fluorescent microscope LSM 510 with an ×63 oil-immersion objective.

RNA Preparation and Real-Time Quantitative PCR

Liver, spleen and duodenum samples were dissected for RNA isolation, rapidly frozen, and stored in liquid nitrogen. Total RNA was extracted and purified using the RNeasy Lipid Tissue kit (Qiagen, Courtaboeuf, France). All primers were designed using the Primer Express 2.0 software (Applied Biosystems, Foster City). Real-time quantitative PCR (Q-PCR) reactions were prepared with M-MLV reverse transcriptase (Promega, Charbonniéres-les-Bains, France) and LightCycler® 480 DNA SYBR Green I Master reaction mix (Roche Diagnostics, Mannheim, Germany) as previously described and run in duplicate on a LightCycler® 480 Instrument (Roche Diagnostics).

Western Blot Analysis

Livers were homogenized in a FastPrep-24 Instrument (MP Biomedicals Europe, Illkirch, France) for 20 sec at 4 m/s. The lysis buffer (50 mM Tris-HCl, pH 8, 150 mM NaCl, 5 mM EDTA, pH 8, 1% NP-40) included inhibitors of proteases (1 mM PMSF, 10 μg/ml leupeptin, 10 mg/ml pepstatin A, and 1 mg/ml antipain) and of phosphatases (10 μl/ml phosphatase inhibitor cocktail 2, Sigma-Aldrich, Saint-Quentin Fallavier, France). Proteins were quantified using the Bio-Rad Protein Assay kit (Bio-Rad Laboratories, Hercules, Calif.) based on the method of Bradford. Protein extracts (30 μg for phospho-Smad and 60 μg for Smad5) were diluted in Laemmli buffer (Sigma-Aldrich), incubated for 5 minutes at 95° C., and subjected to SDS-PAGE. Proteins were then transferred to Hybond-C Extra nitrocellulose membranes (Amersham Biosciences, Orsay, France). Membranes were blocked with Odyssey blocking buffer (LI-COR Biosciences, Lincoln, Nebr.), incubated with a rabbit polyclonal antibody to phosphorylated Smad1/5/8 (1/500, Cell Signaling Technology) or a goat polyclonal antibody to Smad5 (1/200, Santa Cruz Biotechnology, Santa Cruz, Calif.) and a mouse monoclonal antibody to β-actin (1/20000, Sigma-Aldrich) at 4° C. overnight, and washed with PBS-0.1% Tween-20 buffer. Following incubation with infrared IRDye 800 anti-rabbit or anti-goat and IRDye 680 goat anti-mouse secondary antibodies (1/15000, LI-COR Biosciences), membranes were scanned on the Odyssey Infrared Imaging System. Band sizing was performed using the Odyssey 3.0 software (LI-COR Biosciences) and quantification of phospho-Smad activity and of Smad5 was calculated by normalising the specific probe band to β-actin.

Statistical Analyses

Log-transformed values of liver and spleen iron contents were compared by Student's t-tests. The relative expression ratios (and standard errors) of liver, spleen and/or duodenum transcripts between Bmp6^{-/-} mice and wild-type controls or LPS- and saline-treated animals were calculated using the relative expression software tool (REST, http://rest.gene-quantification.info). The mathematical model is based on the mean Cp deviation between sample and control groups of target genes, normalized by the mean Cp deviation of the reference gene beta-glucuronidase. An efficiency correction was performed and randomization tests, that have the advantage of making no distributional assumptions about the data, were used to determine statistical significance.

Results

Hepcidin, a key regulator of iron absorption, binds to the cellular iron exporter ferroportin and induces its endocytosis and proteolysis, preventing release of iron from macrophages or intestinal cells into the plasma. In genetic hemochromatosis, sustained deficiency of hepcidin causes excessive iron absorption from the diet and leads to the deposition of iron in the liver and other tissues, with consequent organ damage and functional failure. Hepcidin expression is controlled by the bone-morphogenetic protein (BMP)-signaling pathway. The signal is initiated when BMP ligands bind and activate receptor serine/threonine kinases at the hepatocyte cell surface. The resulting receptor complex propagates the signal through phosphorylation of cytoplasmic effectors, the receptor-regulated Smads (R-Smads) 1, 5 and 8. Once phosphorylated, the R-Smads form heteromeric complexes with the common mediator Smad4 and translocate to the nucleus, where they modulate target gene transcription. While many BMP ligands, including BMP2, BMP4 and BMP9, can positively regulate hepcidin expression in vitro, the inventors have shown that BMP6 signaling is modulated in response to body iron status and that BMP6 is the endogenous regulators of hepcidin expression and iron homeostasis in vivo.

A functional genomic study in mice fed an iron-enriched or an iron-deficient diet allowed the inventors to show that, in contrast to other Bmp genes, Bmp6 mRNA expression was regulated by iron similarly to hepcidin, and indicated that Bmp6 had a preponderant role in the activation of the Smad signaling pathway leading to hepcidin synthesis in vivo. Of note, Bmp6 is expressed in hepatocytes and its detection by immunochemistry strongly increases in wild-type mice fed an iron-enriched diet. This indicated that iron homeostasis was disturbed in the Bmp6-deficient mice that were derived ten years ago. In contrast to mice deficient for other Bmp molecules, these are viable and fertile. Although there is a delay in ossification strictly confined to the developing sternum in Bmp6 mutant embryos, newborn and adult Bmp6 mutants have skeletal elements indistinguishable from wild-type mice, implying that Bmp6 is not required for normal skeletal development.

The inventors found that targeted disruption of Bmp6 results in massive iron overload. At 7 weeks of age, serum transferrin saturations were close to 100%. Bmp6^{-/-} mice also had 11-fold more non-heme liver iron than had wild-type mice. Conversely, splenic non-heme iron content was decreased in Bmp6^{-/-} mice compared with wild-type controls (FIG. 1).

The inventors further examined the sites of iron accumulation by staining histological sections for iron. At 7 weeks of age, there was considerable iron accumulation in liver parenchymal cells (hepatocytes) of Bmp6^{-/-} mice, whereas iron staining was minimal in Bmp6^{-/-} splenic macrophages. Hepatocellular iron deposition was extending from periportal to centrilobular hepatocytes, following blood flow in the liver (FIG. 2). Iron accumulation was also observed in acinar cells of the exocrine pancreas, in the heart and in renal convoluted tubules.

By 7 weeks of age, Bmp6^{-/-} mice have accumulated significantly more iron than 12-week-old mice of different genetic backgrounds deficient for the classical hemochromatosis gene Hfe. Targeted disruption of murine Bmp6 gene thus results in a mouse severe iron loading phenotype similar to that of mice deficient for the hemojuvelin gene Hjv, for Smad4, or for hepcidin, and to that of human patients with juvenile hemochromatosis. The inventors thus conclude that Bmp6 plays a critical role in the control of iron homeostasis and that mutations in BMP6 might be causing juvenile hemochromatosis. The inventors next examined liver mRNA expression of hepcidin and other genes previously shown to be regulated by iron like hepcidin. They found that Bmp6^{-/-} mice had approximately 22-fold less hepatic hepcidin mRNA than wild-type controls (FIG. 1). Notably, expression of other genes known (Id1, Smad7) or suspected (Atoh8) to be targets of the BMP/Smad signaling pathway was markedly reduced in liver obtained from Bmp6^{-/-} mice.

Since Bmp6 transmits signal through phosphorylation of Smads 1, 5 and 8, the inventors compared the relative abundance of phosphorylated forms of Smad1/5/8 in liver extracts of Bmp6^{-/-} and wild-type mice by western blot analysis. As expected, Smad1/5/8 phosphorylation was significantly reduced in Bmp6^{-/-} mice. They then examined nuclear translocation of phospho-Smad1/5/8 by labelling liver tissue sections with an antibody that detects Smad1, Smad5 and Smad8 when phosphorylated at serines in the carboxy-terminal domain. In Bmp6^{-/-} mice, immunostaining of phosphorylated Smad1/5/8 was weak and distributed evenly in cytoplasm and nucleus, indicating that levels of Smad1/5/8 phosphorylation were low and without significant nuclear translocation, and explaining why hepcidin mRNA levels are markedly reduced in these mice. In contrast, phospho-Smad staining was observed in the hepatocyte nuclei of wild-type mice fed a diet of normal iron content and was strongly induced in those of wild-type animals fed an iron-enriched diet for one week to induce experimental iron overload (FIG. 3).

The dramatic iron accumulation in Bmp6-deficient mice led the inventors to evaluate the expression of genes involved in duodenal iron absorption by real-time quantitative PCR. Transcripts of the brush-border surface ferric reductase Dcytb and the apical transmembrane iron transporter Dmt1 were elevated about 14.6- and 13.8-fold, respectively, while the mRNA of the basolateral membrane transporter ferroportin (Fpn1) was increased about 2.2-fold. This increase may be induced by the lower amount of stainable iron present in proximal duodenal enterocytes in Bmp6^{-/-} mice compared with wild-type controls. The induction of Dmt1 in the duodenum was confirmed by immunohistochemistry. Weak staining of Dmt1 protein was detectable in wild-type controls, but Bmp6^{-/-} mice had intense staining along the brush border (FIG. 4). The inventors also examined ferroportin expression by immunohistochemistry, focusing on tissues where ferroportin is known to be important. Ferroportin is normally expressed at low levels in the absorptive enterocytes lining the intestinal villi. However, in Bmp6^{-/-} mice, the inventors observed a massive increase in ferroportin protein expressed at the basolateral membrane (FIG. 4). Similarly, ferroportin expression was markedly enhanced in tissue macrophages of the livers and spleens of Bmp6^{-/-} mice compared with those of wild-type controls. This is consistent with lack of hepcidin expression in these mice, leading to stabilisation of ferroportin at the membrane of enterocytes and tissue macrophages.

The inventors have also shown that inflammation influences body iron balance. Hepcidin is part of the type II acute phase response and is thought to have a crucial role in anemia of chronic disease. Whereas hepcidin is induced by activation of the inflammatory pathway in Hjv-deficient mice, this induction is not observed in mice with liver-specific Smad4 deficiency. To determine whether lipopolysaccharide (LPS)-dependent induction of hepcidin requires Bmp6, the inventors treated Bmp6 mutant mice and wild-type controls with LPS or 0.9% NaCl. As expected, the acute phase genes II6, Tnf and Crp were strongly induced in LPS-treated mice compared with saline-treated animals. Hepcidin gene expression was induced about 24-fold in response to the LPS treatment in Bmp6^{-/-} mice and 2.6-fold in wild-type controls (FIG. 1). Interestingly, hepcidin levels in LPS-injected Bmp6^{-/-} animals do not reach those of wild-type controls. These findings indicate that the total level of hepcidin expression observed upon inflammation is additive to the baseline level and argue for the existence of two independent pathways that lead to the regulation of hepcidin expression. Of these two pathways, only the iron-sensing pathway requires functional Bmp6 and Hjv. It is not clear yet how LPS activates hepcidin production in Bmp6-deficient mice, although there is evidence supporting a role for BMP/TGFβ signaling. Indeed, transcriptional activation of hepcidin by IL-6 is abrogated in mice with liver-specific conditional knockout of Smad4 and chemical inhibition of BMP signal transduction in a human hepatoma cell line blocks not only the induction of hepcidin expression by BMPs but also by IL-6. Furthermore, a BMP-responsive element in the hepcidin promoter is required to control hepatic expression in response to IL-6. Further work is needed to identify which TGF-β/BMP superfamily ligands, other than BMP6, function as endogenous activators of hepcidin expression during inflammation.

BMP molecules were initially identified by their capacity to induce endochondral bone formation. However, mild and/or extremely localized skeletal defects are observed in mice deficient for Bmp1, Bmp2, Bmp4 and Bmp7, which contrasts strongly with profound and specific effects on gut, heart, neural tube or kidney morphogenesis. Physiological actions of BMPs in soft tissues thus appear more important than their actions in the skeleton. Bmp6^{-/-} mice are notable for the absence of skeletal defects but, curiously, no effect of Bmp6 deficiency on other tissues or organs has been reported so far. The inventors have shown here for the first time a previously unsuspected but essential role of Bmp6 in the maintenance of iron homeostasis. Although other Bmp molecules are functional in the severely iron overloaded Bmp6-deficient mice, they do not compensate for the absence of Bmp6, demonstrating that this novel iron-regulatory function for the family of BMP molecules is unique to Bmp6. The iron burden in Bmp6 mutant mice is significantly more severe than in Hfe-deficient mice, and closely resembles disorders observed in Hjv-, hepcidin- or Smad4-deficient mice. This indicates that the human BMP6 gene is a candidate locus in those patients with severe juvenile hemochromatosis not attributable to hemojuvelin or hepcidin. Individuals with mutations in HFE, transferrin receptor 2 (TFR2) and hemojuvelin (HJV) have low hepcidin levels, and consequently, they are unable to effectively repress iron absorption. While the mechanisms by which TFR2 and HFE regulate hepcidin remain enigmatic, HJV was shown to be a cell-surface BMP co-receptor and to augment signal transduction through this pathway. Interestingly, while soluble hemojuvelin inhibits induction of hepcidin by several BMP ligands in vitro, careful examination of the data shows that this inhibition is far more efficient for BMP6. These results indicate that HJV is a co-receptor for BMP6 in vivo and that BMP6 and HJV act coordinately to induce hepcidin expression.

### Example 2

### BMP/Smad Signaling is not Enhanced in Hfe-Deficient Mice Despite Increased Bmp6 Expression

Summary

Impaired regulation of hepcidin expression in response to iron loading appears to be the pathogenic mechanism for hereditary hemochromatosis. Iron normally induces expression of the BMP6 ligand which, in turn, activates the BMP/Smad signaling cascade directing hepcidin expression. The molecular function of the HFE protein, involved in the most common form of hereditary hemochromatosis, is still unknown. The inventors have used Hfe-deficient mice of different genetic backgrounds to test whether HFE has a role in the signaling cascade induced by BMP6. At 7 weeks of age, these mice have accumulated iron in their liver and have increased Bmp6 mRNA and protein. However, in contrast to mice with secondary iron overload, levels of phosphorylated Smads 1/5/8 and of Id1 mRNA, both indicators of BMP signaling, are not significantly higher in the liver of these mice than in wild-type livers. As a consequence, hepcidin mRNA levels in Hfe-deficient mice are similar or marginally reduced, compared with 7-week-old wild-type mice. The inappropriately low levels of Id1 and hepcidin mRNA observed at weaning further suggest that Hfe-deficiency triggers iron overload by impairing hepatic Bmp/Smad signaling. HFE therefore appears to facilitate signal transduction induced by the BMP6 ligand.

Materials and Methods

Mice

Hfe-deficient mice on the C57BL/6 (B6) and DBA/2 (D2) backgrounds were derived as previously described (Bensaid M et al., Multigenic control of hepatic iron loading in a murine model of hemochromatosis, Gastroenterology 2004; 126:1400-1408). They were maintained at the IFR30 animal facility, as well as wild-type controls of the same genetic backgrounds. All experiments were performed on males. Unless otherwise specified, mice received a standard rodent diet (200 mg iron/kg body weight; SAFE, Augy, France) and were killed at 7 weeks. Experimental iron overload was obtained by feeding 4-week-old B6 and D2 wild-type mice the same diet supplemented with 8.3 g/kg carbonyl iron (Sigma-Aldrich, Saint Quentin Fallavier, France) for three weeks. Three-week-old Hfe-deficient mice and litter-matched wild-type controls were obtained from B6D2F1 heterozygous (Hfe^{+/-}) parents. Experimental protocols were approved by the Midi-Pyrénées Animal Ethics Committee.

Tissue Iron Measurement

Quantitative measurement of hepatic non-heme iron was performed as described previously. Results are reported as micrograms of iron per gram dry weight of tissue.

RNA Preparation and Real-Time Quantitative PCR

Liver samples were dissected for RNA isolation, rapidly frozen, and stored in liquid nitrogen. Total RNA was extracted and purified using the RNeasy Lipid Tissue kit (Qiagen, Courtaboeuf, France). All primers were designed using the Primer Express 2.0 software (Applied Biosystems, Foster City). Real-time quantitative PCR (Q-PCR) reactions were prepared with M-MLV reverse transcriptase (Promega, Charbonniéres-les-Bains, France) and LightCycler® 480 DNA SYBR Green I Master reaction mix (Roche Diagnostics, Mannheim, Germany) and run in duplicate on a LightCycler® 480 Instrument (Roche Diagnostics).

Immunohistochemistry

Four-micrometer sections of paraffin-embedded tissues were mounted on glass slides. Antigen retrieval was performed by incubating tissue sections with trypsin (1 mg/ml) for 8 min at 37° C. Endogenous peroxidase activity was quenched by incubating specimens with Dako REAL Peroxidase Blocking Solution (Dako, Trappes, France). Tissue sections were then blocked with normal horse blocking serum (Vector Laboratories, Burlingame, Calif.) and incubated 1 h at RT with the primary anti-BMP6 (N-19) antibody (1/100; Santa Cruz Biotechnology, Santa Cruz, Calif.) diluted in PBS-1% BSA and 1% FCS. Immunohistochemical staining was performed using the ImmPRESS Reagent (ImmPRESS Anti-Goat Ig peroxidase Kit; Vector Laboratories) according to the instructions of the manufacturer. Sections were counterstained with hematoxylin. Tissue sections from Bmp6-deficient mice were used to test antibody specificity.

Western Blot Analysis

Livers were homogenized in a FastPrep-24 Instrument (MP Biomedicals Europe, Illkirch, France) for 20 sec at 4 m/s. The lysis buffer (50 mM Tris-HCl, pH 8, 150 mM NaCl, 5 mM EDTA, pH 8, 1% NP-40) included inhibitors of proteases (1 mM PMSF, 10 μg/ml leupeptin, 10 mg/ml pepstatin A, and 1 mg/ml antipain) and of phosphatases (10 μl/ml phosphatase inhibitor cocktail 2, Sigma-Aldrich, Saint-Quentin Fallavier, France). Proteins were quantified using the Bio-Rad Protein Assay kit (Bio-Rad Laboratories, Hercules, Calif.) based on the method of Bradford. Protein extracts (30 μg for phospho-Smad and 60 μg for Smad5) were diluted in Laemmli buffer (Sigma-Aldrich), incubated for 5 minutes at 95° C., and subjected to SDS-PAGE. Proteins were then transferred to Hybond-C Extra nitrocellulose membranes (Amersham Biosciences, Orsay, France). Membranes were blocked with Odyssey blocking buffer (LI-COR Biosciences, Lincoln, Nebr.), incubated with a rabbit polyclonal antibody to phosphorylated Smad1/5/8 (1/500, Cell Signaling Technology; lot 8) or a goat polyclonal antibody to Smad5 (1/200, Santa Cruz Biotechnology, Santa Cruz, Calif.) and a mouse monoclonal antibody to β-actin (1/20000, Sigma-Aldrich) at 4° C. overnight, and washed with PBS-0.1% Tween-20 buffer. Following incubation with infrared IRDye 800 anti-rabbit or anti-goat and IRDye 680 anti-mouse secondary antibodies (1/15000, LI-COR Biosciences), membranes were scanned on the Odyssey Infrared Imaging System. Band sizing was performed using the Odyssey 3.0 software (LI-COR Biosciences) and quantification of phosphorylated Smads and of Smad5 was calculated by normalizing the specific probe band to β-actin.

Statistical Analyses

Log-transformed values of liver iron contents were compared by Student's t-tests. The relative expression ratios (and standard errors) of liver transcripts between Hfe^{-/-} mice and wild-type controls were calculated using the relative expression software tool (REST, http://rest.gene-quantification.info). The mathematical model is based on the mean crossing point (Cp) deviation between sample and control groups of target genes, normalized by the mean Cp deviation of the reference gene Hprt. An efficiency correction was performed and randomization tests, that have the advantage of making no distributional assumptions about the data, were used to determine statistical significance.

Results

Hfe-Deficiency Promotes Liver Expression of Bmp6

As previously observed, whereas 7 week-old Hfe-deficient mice of the DBA/2 (D2) background have a higher liver iron burden than Hfe-deficient mice of the C57BL/6 (B6) strain, wild-type mice of the B6 background fed an iron-enriched diet for three weeks are reproducibly more heavily iron-loaded than wild-type D2 mice fed the same iron-rich diet (FIG. 5). This reflects differences in the genetic susceptibility to iron-loading in the presence or absence of functional Hfe. Real-time quantitative PCR shows that expression of Bmp6 is significantly up-regulated not only in the liver of wild-type mice with secondary iron overload but also in the liver of Hfe-deficient mice compared with that of wild-type controls (FIG. 5). Noticeably, mice with the highest hepatic iron burden (B6 mice with secondary iron overload and Hfe-deficient D2 mice) have the highest induction of Bmp6 relative to control animals. The inventors thus examined liver expression and cellular localization of Bmp6 by immunohistochemistry, using an antibody raised against a peptide mapping within the internal region of BMP6. Enhanced Bmp6 staining was observed in Hfe-deficient mice and in wild-type mice with secondary iron overload. Interestingly, the distribution of Bmp6 in the liver is zonal and, unlike iron deposits that are periportal (FIG. 6A), Bmp6 staining is centrilobular (FIG. 6B). This centrilobular layout of Bmp6 is observed in both Hfe-deficient mice and wild-type mice with secondary iron overload. BMP6 expression was previously shown to be confined to nonparenchymal liver cells, namely hepatic stellate cells and Kupffer cells. However, in iron-loaded livers, Bmp6 is also found in the hepatocytes, noticeably at the basolateral membrane domain as previously reported for hemojuvelin and TFR2 (FIG. 6C-D). This staining was not observed in Bmp6-deficient mice or with control goat IgG.

Smad1/5/8 Phosphorylation is not Increased in Hfe-Deficient Mice

Since Bmp6 transmits signal through phosphorylation of Smads, we tested whether phosphorylation of Smad1/5/8 was increased in liver extracts of Hfe^{-/-} mice. Total protein lysates from three groups of animals were obtained for the two strains B6 and D2: (i) wild-type controls fed a standard rodent diet; (ii) Hfe^{-/-} mice fed the same standard rodent diet; and (iii) wild-type controls fed an iron-enriched diet to induce secondary iron overload. The amount of the phosphorylated forms of Smad1/5/8 in each group was determined by western blot analysis. As shown on FIG. 7, while the iron-enriched diet induced Smad1/5/8 phosphorylation in both strains, no significant increase in Smad1/5/8 phosphorylation was observed in 7 week-old B6 or D2 Hfe^{-/-} mice compared with wild-type controls. Therefore, Hfe^{-/-} mice do not appropriately respond to the increase in Bmp6. We also measured the levels of Id1 mRNA in the liver of the different mice. Id1 is a direct target gene for BMPs and phosphorylated Smads 1 and 5 have been shown to regulate its transcription through direct binding to specific elements on its promoter. Its up-regulation therefore is an indicator of activation of the Bmp signaling cascade. As can be seen in FIG. 5, whereas Id1 mRNA expression is very significantly up-regulated in the livers of mice with secondary iron overload, no such up-regulation is seen in the livers of Hfe-deficient mice, despite the increase in Bmp6 liver expression.

Up-Regulation of Bmp6 is Preceded by a Marked Down-Regulation of Hepcidin Expression

Because phosphorylation of Smad proteins 1/5/8 was not significantly different between 7-week-old Hfe-deficient mice and wild-type controls, the inventors expected that hepcidin transcription would also be similar in the two groups of animals. Indeed, as shown on FIG. 5, the inventors found that Hamp mRNA levels in Hfe^{-/-} mice of the B6 genetic background were equivalent to those in wild-type mice, and only slightly reduced in Hfe^{-/-} mice of the D2 background. The excessive iron burden observed in 7-week-old Hfe-deficient mice is difficult to reconcile with quasi normal levels of hepcidin. This led the inventors to hypothesize that iron overload in 7-week-old Hfe-deficient mice results from reduced hepcidin production earlier in life. To test this hypothesis, the inventors quantified liver iron as well as Bmp6 and Hamp mRNA levels in 3-week-old Hfe-deficient mice and wild-type controls. Weaning from a low-iron diet (milk) to the relatively high-iron diet provided by chow is associated with a rapid increase in transferrin saturation and in hepcidin expression within one week. The inventors suspected that this increase would be influenced by Hfe and therefore used Hfe-deficient mice and litter-matched controls to ensure that they were carefully matched on the age. As can be seen on FIG. 8, at 3 weeks of age, Hfe-deficient mice have liver iron content and Bmp6 gene expression similar to wild-type animals. However, their Hamp gene expression is about 8-fold lower than in control mice. This indicates that down-regulation of hepcidin expression is the first biological manifestation of Hfe-deficiency and precedes liver iron accumulation and increase in Bmp6 expression. Interestingly, although the inventors were unable to detect a statistically significant decrease in Smad1/5/8 phosphorylation by western-blot analysis, the levels of Id1 mRNA, an indicator of activation of the BMP signaling cascade, are reduced by about 50% in these young Hfe-deficient mice compared with wild-type controls, further suggesting that Bmp6 signaling is impaired by lack of functional Hfe. In wild-type mice fed an iron-enriched diet or an iron-deficient mice, modulation of Smad1/5/8 phosphorylation is always less pronounced than modulation of Id1 mRNA, which is itself often less pronounced than modulation of Hamp mRNA. Therefore, the inventors cannot exclude an amplification of the response to Bmp6 between Smad1/5/8 phosphorylation and the transcription of the specific targets. Given that there is only a two-fold decrease in Id1 mRNA expression in 3 week-old Hfe-deficient mice compared with wild-type mice (as seen in FIG. 8), it is possible that modulation of Smad1/5/8 phosphorylation in these mice is too low to be visualized by western blot analyses.

### CONCLUSION

Although the site of HFE regulatory function is the hepatocyte, the exact mechanisms by which HFE regulates iron homeostasis remain elusive. The inventor's data indicate that lack of functional Hfe early in life severely impairs the Bmp/Smad signaling cascade, resulting in the downregulation of hepcidin observed in 3 week-old mice in this and previously reported studies. As a consequence, there is no feed-back mechanism to limit iron efflux from intestinal enterocytes. Between 3 and 7 weeks of age, Hfe-deficient mice progressively accumulate iron and, interestingly, retain their ability to increase Bmp6 in response to body iron excess, as do mice with secondary iron overload or mice with genetic iron overload due to inactivation of the Smad4 or the Hamp gene. However, due to the lack of functional Hfe, the response to increased Bmp6 expression is blunted compared with that of mice with secondary iron overload and only reaches levels observed in wild-type controls fed a standard rodent diet. Given their iron burden, Smad1/5/8 phosphorylation, Id1 and hepcidin expression are all inappropriately low in 7 week-old Hfe-deficient mice. The age-related changes in Bmp6 and Hamp expression observed here in Hfe-deficient mice explain why, several weeks after birth, intestinal iron absorption decreases and hepatic iron concentrations reach a plateau. Of note, although Hfe-deficient D2 mice have higher Bmp6 gene expression than Hfe-deficient B6 mice (p=0.001), they have slightly less hepcidin mRNA. Genetically determined differences in the maturation, secretion or inhibition of Bmp6 between strains may affect the efficacy of signal transduction and explain these variations.

In hemochromatosis patients iron absorption also declines as the iron load increases. Furthermore, hepcidin concentrations in the sera of iron-loaded patients with HH resulting from mutations in the HFE gene are similar to controls, indicating a disease time course similar to that observed in mice although more spread out over time. Interestingly, hepcidin concentrations are lower than controls in patients who have been iron-depleted by phlebotomy treatment. BMP6 levels are high in untreated patients and therapeutic venesections, by removing excess iron stores, restore these levels to those seen in controls, thus reducing the efficacy of signal transduction. The consequent decrease in hepcidin expression then explain the re-accumulation of iron in the absence of maintenance phlebotomies.

The inventors demonstrated that HFE impairs propagation of the signaling cascade induced by the BMP6 ligand and suggests that HFE and the BMP type I and II serine/threonine kinase receptors are associated at the hepatocyte cell membrane and that this association is required to ensure proper signal transduction. Hemojuvelin, TFR2 and BMP6 all localize to the hepatocyte basolateral membrane domain, which indicates a functional interaction of these molecules in the context of iron metabolism regulation. HFE, TFR2 and other proteins like BMP6, its receptors and hemojuvelin then form in this functional membrane domain an iron signaling complex that induces hepcidin transcription via Smad proteins. Interestingly, there are previous reports of physical associations of MHC-class I molecules to tetrameric membrane receptors like the insulin receptor in mouse liver membranes.

In summary, the inventors demonstrated that the role of HFE is not solely limited to iron sensing by a mechanism involving a competition between HFE and diferric transferrin for TFR1 binding. Indeed, the inventors showed that HFE is necessary for correct signal transduction from BMP6, suggesting that, when dissociated from TFR1, HFE participates in the BMPRI/II molecular complex. In the presence of HFE, basal levels of BMP6 are probably sufficient for physiologic modulation of hepcidin outside of massive iron overload. Indeed, wild-type D2 mice fed an iron-enriched diet for a short period have increased transferrin saturation and elevated hepcidin expression, but no increase in hepatic iron or in Bmp6 mRNA expression. Furthermore, at weaning from milk to the relatively high-iron diet provided by chow, wild-type mice have a rapid increase in transferrin saturation and in hepcidin expression, but again no increase in hepatic iron or in Bmp6 mRNA expression. Therefore, the ability to increase Bmp6 expression seems restricted to animals with liver iron accumulation, whether due to Hfe-deficiency or to an iron-enriched diet for several weeks. A greater amount of the Bmp6 ligand then allows a more efficient propagation of the signaling cascade which clearly improves the status of Hfe-deficient animals and hopefully that of hemochromatosis patients. This explains why a plateau in iron loading is reached over time and why hepcidin decreases after iron depletion in human patients.

### REFERENCES

Throughout this application, various references describe the state of the art to which this invention pertains. The disclosures of these references are hereby incorporated by reference into the present disclosure.

1. A method for diagnosing an iron dysregulation in a subject comprising the step of measuring the level of BMP6 in a body fluid.

2. The method according to claim 1, wherein said body fluid is selected from the group consisting of whole blood, blood plasma, serum and urine obtained from said subject.

3. A method for preventing iron accumulation in a subject, comprising the step of administering to said subject:
an effective amount of BMP6, a fragment or a derivative thereof, said fragment or derivative inducing hepcidin expression; or
an effective amount of a vector comprising a nucleic acid coding for BMP6, a fragment or a derivative thereof, said fragment or derivative inducing hepcidin expression.

4. The method according to claim 3, wherein said subject is predisposed to iron overload.

5. A method for preventing iron reaccumulation in a subject who has been iron depleted, said method comprising the step of administering to said subject:
an effective amount of BMP6, a fragment or a derivative thereof, said fragment or derivative inducing hepcidin expression; or
an effective amount of a vector comprising a nucleic acid coding for BMP6, a fragment or a derivative thereof, said fragment or derivative inducing hepcidin expression.

6. A method for treating a subject suffering from iron deficiency, comprising the step of administering to said subject an effective amount of an inhibitor of BMP6 induction of hepcidin expression.

7. The method according to claim 6, wherein said inhibitor of BMP6 induction of hepcidin expression is an agent downregulating BMP6 expression.

8. The method according to claim 7, wherein said agent downregulating BMP6 expression comprises a nucleic acid which interferes with the expression of BMP6.

9. The method according to claim 6, wherein said inhibitor of BMP6 induction of hepcidin expression is an antibody against BMP6 or a fragment or derivative thereof, said fragment or derivative inhibiting BMP6 induction of hepcidin expression.

10. A medicament comprising an inhibitor of BMP6 induction of hepcidin expression together with a pharmaceutically acceptable carrier.

11. A method for diagnosing an autosomal recessive hereditary pathology, or a risk of an autosomal recessive hereditary pathology, in a subject, said method comprising the step of detecting a defective mutation in the BMP6 gene in a sample obtained from said subject, wherein the presence of homozygosity or compound heterozygosity for BMP6 mutations is indicative of an autosomal recessive pathology or a risk of an autosomal recessive hereditary pathology.

12. The method according to claim 11, wherein said defective mutation in the BMP6 gene is a mutation which results in a reduction of BMP6 expression or in impaired binding to type 1 and type 2 receptors.
